(19) United States
(12) Patent Application Publication
(10) Pub. No.: US 2012/0115212 A1
(43) Pub. Date: May 10, 2012

(54) Assay Tube

(76) Inventors: Bernhard WEIGL, Seattle, WA (US); Jay L. GERLACH, Seattle, WA (US); Paul D. LABARRE, Seattle, WA (US)

(21) Appl. No.: 12/256,056

(22) Filed: Oct. 22, 2008

Related U.S. Application Data
(60) Provisional application No. 60/981,780, filed on Oct. 22, 2007.

(51) Int. Cl. C12M 1/40

(52) U.S. Cl. 435/287.2

(57) ABSTRACT

A non-instrumented biochemical assay device, such as a device for the processing of clinical specimens for molecular diagnostic applications. In one embodiment, an assay device comprises a lid which covers a reagent tube, which is disposed on a base. The platform utilizes chemical heating and optional temperature modulation by phase change material to prepare the sample and to amplify the target nucleic acid using Loop-Mediated Isothermal Amplification. The device features a simple indicator for a positive reaction, for example turbidity or fluorescence.

### CROSS REFERENCE TO RELATED APPLICATIONS

This application claims the benefit of U.S. Provisional Application No. 60/981,780, filed on Oct. 22, 2007, which is hereby incorporated by reference in its entirety.

### BACKGROUND OF THE INVENTION

1. Field of the Invention

The present invention generally relates to assay devices, more specifically to non-instrumented biochemical assay devices, such as a device for the processing of clinical specimens for molecular diagnostic applications.

2. Background Art

Over the past two decades, assays based on nucleic-acid (NA) amplification (e.g., polymerase chain reaction (PCR) assays and loop-mediated isothermal amplification (LAMP) assays) have found many applications in medical diagnostics, forensics, biodefense, biotechnological research, and other fields. Such assays detect, for example, the presence of a pathogen through a process of extracting relevant genetic material, amplifying (multiplying) pathogen-specific genetic material, and then observing the amplified signal. Extraction of genetic material typically requires a technician to prepare the sample through several steps. The amplification step is usually achieved through (PCR), a process that requires a mechanism to heat and cool the sample through many cycles. These first two steps (i.e. extracting and amplifying), in particular, present significant challenges to creating small, easy-to-use tests that do not require technical skill and laboratory support. Despite several interesting attempts to develop small, portable NA assays, the vast majority remain lab-based and require complex sample preparation as well as an instrument for heating (and/or heat-cycling) and detection.

The high-tech, high-cost centralized laboratories necessary to practice conventional NA assay techniques are found almost exclusively in the developed world. Where such centralized laboratories exist at all, they are in urban areas, catering primarily to the more affluent segments of urban society. In contrast, health care facilities in rural areas commonly have only basic equipment, health workers with less training, and little or no resources to maintain equipment and handle reagents. This is especially true in developing countries. Therefore, there is a need in the art for development of a NA assay platform that requires absolutely no instrument, power, or external reagents.

### BRIEF SUMMARY OF THE INVENTION

In one embodiment of the present invention, an assay platform has a main body with a first end and a second end. A first cap is detachably connected to the first end of the main body. The first cap has a first exothermic chemical heater. A second cap is detachably connected to the second end of the main body. The second cap has a second exothermic chemical heater. The main body also has a first compartment for sample preparation and a second compartment for nucleic-acid amplification.

In another embodiment of the present invention, an assay platform has a main body with a first end and a second end. A cap is detachably connected to the first end of the main body. The cap has an exothermic chemical heater and phase change material. The main body has a first compartment for sample preparation and a second compartment for nucleic-acid amplification. The exothermic chemical heater and phase change material provide a first temperature for a first time interval corresponding to a sample preparation temperature and time interval. The exothermic chemical heater and phase change material provide a second temperature for a second time interval corresponding to a nucleic-acid amplification temperature and time interval.

In another embodiment of the present invention, an assay platform has a main body having a first compartment for sample preparation and a second compartment for nucleic-acid amplification. The main body has a first phase change material characterized by a first phase change temperature at least partially surrounding the first compartment. The main body has a second phase change material characterized by a second phase change temperature at least partially surrounding the second compartment. There is an exothermic chemical heater in thermal contact with said first and second phase change materials.

### BRIEF DESCRIPTION OF THE DRAWINGS/FIGURES

The accompanying drawings, which are incorporated herein and form a part of the specification, illustrate the present invention and, together with the description, further serve to explain the principles of the invention and to enable a person skilled in the pertinent art to make and use the invention.

FIG. 1 is perspective view of an assay device according to the present invention shown with the top removed;

FIG. 2 is a cross sectional view of the assay device of FIG. 1;

FIG. 3 is perspective view of the assay device of FIG. 1 shown with the top attached; the device is in a first configuration;

FIG. 4 is perspective view of the device of FIG. 3 in a second configuration;

FIG. 5 is a cross sectional view of the device of FIG. 4;

FIG. 6 is an exploded perspective view of the assay device of FIGS. 1-5;

FIG. 7 is an exploded schematic view of an embodiment of an assay device according to the present invention;

FIG. 8 is a schematic of the assay device of FIG. 7, shown at the completion of a positive diagnostic test; and

FIG. 9 is an exploded cross sectional view of an assay device according to the present invention.

### DETAILED DESCRIPTION OF THE INVENTION

The present invention is now described with reference to the figures where like reference numbers indicate identical or functionally similar elements. While specific configurations and arrangements are discussed, it should be understood that this is done for illustrative purposes only. A person skilled in the relevant art will recognize that other configurations and arrangements can be used without departing form the spirit and scope of the invention.

The present invention combines exothermic chemical heating and loop-mediated isothermal amplification in a self-contained, easy to use assay device, shown generally at 10. Device 10 can be, for example, a tube-like device. As another example, device 10 can be a vial. Exothermic heating can be applied to provide a self-contained heat source for both the sample preparation step and the amplification step (e.g., LAMP). LAMP is mentioned by way of example, and not by way of limitation. Other types of NA amplification known in the art are suitable for the present invention.

Unlike PCR, LAMP does not require heat cycling. A more detailed explanation of PCR is given in U.S. patent application Ser. No. 12/134,965, filed Jun. 6, 2008, titled “CHEMICAL TEMPERATURE CONTROL,” the disclosure of which is incorporated herein by reference in its entirety. Instead, LAMP requires only a constant temperature, approximately 65° C. (preferably at 62.5° C.). This temperature can vary somewhat, as is known in the art. Furthermore, complex samples, for example whole blood, can be prepared for LAMP amplification simply through mixture with a lysis buffer (which breaks down cell walls) and heating at 99° C. The LAMP amplification temperature can also vary within acceptable tolerances, as is known in the art.

Exothermic materials can generate the needed heat fluxes by themselves, and when combined with phase change material(s) the temperature can be held constant at the desired level(s) for a desired time(s). A more detailed discussion of chemical temperature regulation using exothermic and/or endothermic reactions, phase change materials, and exothermic phase change materials is given in U.S. patent application Ser. No. 12/134,965, filed Jun. 6, 2008, titled “CHEMICAL TEMPERATURE CONTROL,” the disclosure of which is incorporated herein by reference in its entirety As will be explained in more detail, the heat mixtures (one for the sample preparation at approximately 99° C., and one for the LAMP amplification at approximately 65° C., preferably at 62.5° C.) will be contained in caps that interface with the assay device (e.g., tube-like reactor device 10) prepared with reagent and sample. Successful amplification of the target genetic sequence with LAMP results in a visible output, for example, turbidity or fluorescence, and thus no further detection device is needed. Device 10 combines all three steps (1. sample preparation, 2. amplification, and 3. detection) into one compact, inexpensive, and easy to use platform.

Loop-Mediated Isothermal Amplification

As mentioned, LAMP is merely exemplary and is but one of several NA amplification techniques known in the art which are applicable to the present invention. A typical loop-mediated isothermal amplification (LAMP) reaction involves heating the reaction mixture to approximately 95° C. for 5 minutes, followed by a 60-minute incubation at approximately 65° C., preferably at 62.5° C. However, these temperatures and times can vary, allowing for flexibility in engineering applications of LAMP technology. Unlike other isothermal nucleic-acid amplification technologies known in the art (e.g., nucleic acid sequence based amplification, “NASBA,” transcription mediated amplification, “TMA,” or strand displacement amplification, “SDA”), LAMP relies on a single enzyme: DNA polymerase. Sensitivity is comparable to PCR, and LAMP is less prone to nonspecific amplification. Although these other isothermal NA amplification technologies may require more than one enzyme, they may nonetheless be used in the present invention instead of LAMP.

LAMP assays can be used to detect the many diseases, for example, tuberculosis, “TB,” severe acute respiratory syndrome, “SARS,” Mumps, West Nile Virus and Influenza A subtyping, as well as many other human pathogens. By way of example, and not by way of limitation, LAMP can be used to test for malaria. Blood is heated to approximately 99° C. then added directly to the LAMP reaction mix. As amplification progresses a white precipitate forms which is visible to the naked eye. Thus, one can perform visual detection of amplification in the reaction tube with no additional processing. Due to the simplicity of sample processing, isothermal amplification, and visual detection, this assay lends itself to adaptation to a single-use, point-of-care test utilizing exothermic chemical reactions to provide the necessary heat.

Chemical Temperature Regulation

The present invention uses exothermic reactions in combination with phase change materials (PCMs) to provide temperature-controlled heat for performing nucleic-acid amplification assays. The use of chemical reactions can provide significant and controllable amounts of heat. The phase change properties of various substances can be utilized to maintain a steady temperature. In these PCMs a specific amount of energy, the enthalpy of fusion, can transfer into and from the material while the material is at the phase transition temperature. In one embodiment, PCMs can be made from a paraffin. In another embodiment, PCMs can be made from metals or alloys.

Exothermic materials can be activated in various ways. For example, fine iron powder generates heat as soon as it is exposed to moist air and starts oxidizing, whereas a supersaturated sodium acetate trihydrate solution generates heat after crystallization can be triggered through shock waves caused by a physical process such as the sharp and sudden bending of a plastic rod. Both these mechanisms, as well as any other mechanism known in the art, can easily be implemented and triggered in the disposable device 10 shown below.

Example reagents for heating chemical reactions include (1) the catalyzed oxidation of iron (providing up to 69° C.), (2) reaction of calcium oxide and diluted hydrochloric acid (providing >100° C.), (3) reaction of magnesium and copper sulfate which can generate similar temperatures, and (4) sodium acetate, which can generate temperatures suitable for reverse transcription in the 45° C.-55° C. range. As would be apparent to one of skill in the art, other known exothermic heat sources could also be used such as, for example, the heat producing compositions disclosed in U.S. Pat. No. 5,935,486, which is incorporated herein by reference. For example, possible exothermic reactions include (but are not limited to): supercooled liquids, exothermic compounds, and heat of solution. Any of these methods, or others known in the art, can be used as an “exothermic chemical heater.”

Chemical and heat of solution technologies such as mixing of inorganic salts and water that produces an exothermic reaction lend themselves to the generation of heat to reach a desired temperature in compact sizes for diagnostic applications. One of the advantages of utilizing this technology is that it does not require combustion since the act of mixing two substances results in the increase of the temperature of the end-product (in an adiabatic container) for either the dissolving of one compound in the solvent (with a positive heat of solution) or an exothermic chemical reaction.

If a solute dissolves in a solvent without heat of reaction, heat is normally absorbed by the environment. If the dissolutuion occurs adiabatically, normally the temperature falls (heat of solution). When the solute crystallizes out of the solution, the temperature rises. The dissolution of an anhydrous salt (e.g., magnesium sulfate) at its hydrate salt temperature formation may lead to the release of energy owing to the exothermic nature of the hydration.

An exemplary PCM to moderate temperatures at 94° C.+/−3° C. (useful in, for example, the sample preparation step of LAMP) is known by the trademark CERROSHIELD®, which is available from Cerro Metal Products Company, Bellefonte, Pa. It is an inexpensive alloy with a high melting enthalpy per unit volume and a phase transition temperature of 95° C. CERROSHIELD® is approximately 52.5% Bismuth, 32.0% Lead, and 15.5% Tin. An exemplary PCM to moderate temperatures useful for NA amplification is RUBITHERM® RT 65, which is available from Rubitherm Technologies GmbH of Berlin, Germany, and has a melting point of approximately 65° C. There are a wide variety of PCMs available which may be suitable to any choice of NA amplification (e.g., LAMP, PCR, NASBA, TMA, SDA, and the like). A more detailed discussion of chemical temperature regulation using exothermic or endothermic reactions, phase change materials, and exothermic phase change materials is given in U.S. patent application Ser. No. 12/134,965, filed Jun. 6, 2008, titled “CHEMICAL TEMPERATURE CONTROL,” the disclosure of which is incorporated herein by reference in its entirety.

The present invention may include a thermally controlled valve in lieu of (or in addition to) PCMs in order to control temperatures. In one embodiment, the thermally controlled valve can be a self-regulating valve. A self-regulating valve controls the degree to which the reagents of an exothermic chemical reaction come into contact with one another, which in turn regulates the heat that they produce. The self-regulating valve may inhibit contact between these reagents when the temperature rises, thereby maintaining the temperature in a defined range. In one embodiment, the self-regulating valve may expand as the temperature increases, thereby blocking a channel between chemical reagents in order to inhibit further temperature increases. Conversely, the self-regulating valve contracts when cooled, thereby unblocking the channel to allow more chemical reagents to react, which causes the temperature to once again increase. In this way, a defined temperature range may be maintained without the need for complicated and expensive feedback and control systems. The geometry, size, and material of the self-regulating valve may be tailored to any desired temperature range. In one embodiment, a gel that expands with temperature may be used. In another embodiment, a bimetal valve may be used.

### Example Embodiment

### Tube Assay

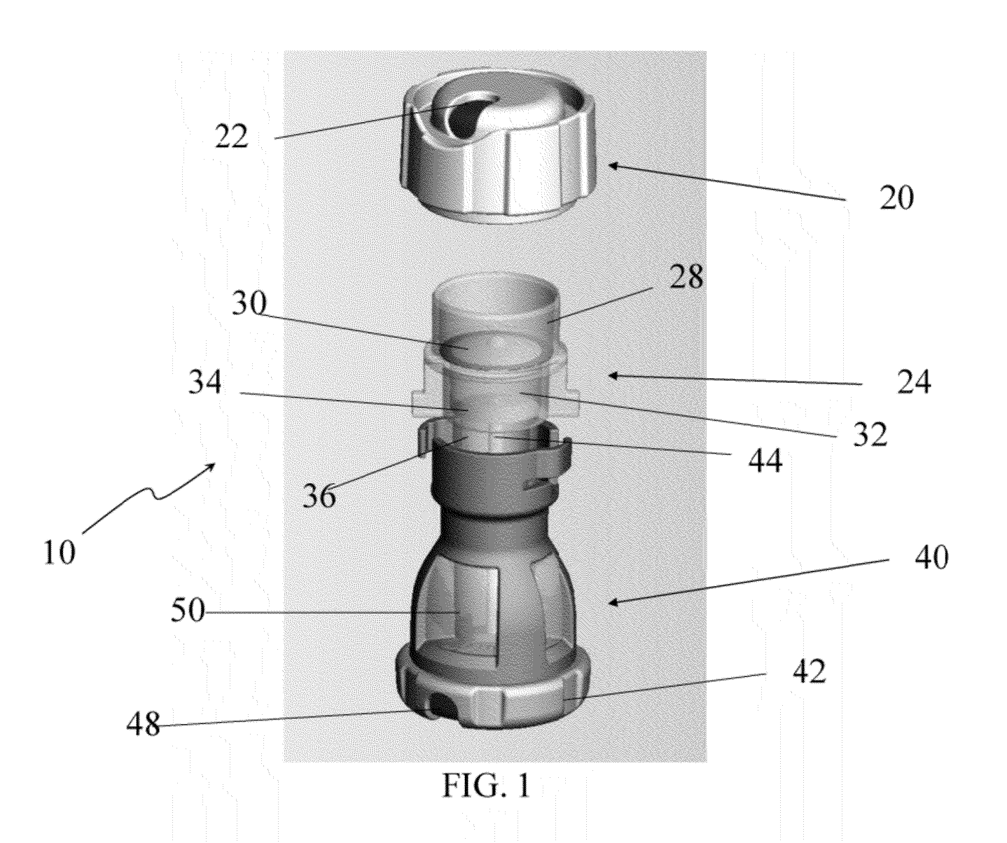

As shown in FIGS. 1-6, device 10 combines the steps of sample preparation, NA amplification, and visual detection of amplification. In one embodiment, the NA amplification can be accomplished using LAMP; however, the present invention is not limited to LAMP. In one embodiment, device 10 can be tube-like. In another embodiment, device 10 can be a vial. Device 10 is not limited to being tube-like or being a vial; rather, device 10 can be any form capable of holding a sample. In one embodiment, device 10 can be approximately the size of a centrifuge tube. Device 10 combines the ease of use of a lateral flow immunoassay (strip test), with the sensitivity of a PCR assay. Device 10 can be made of inexpensive material (e.g., plastic) and can be made with any technique as is known in the art.

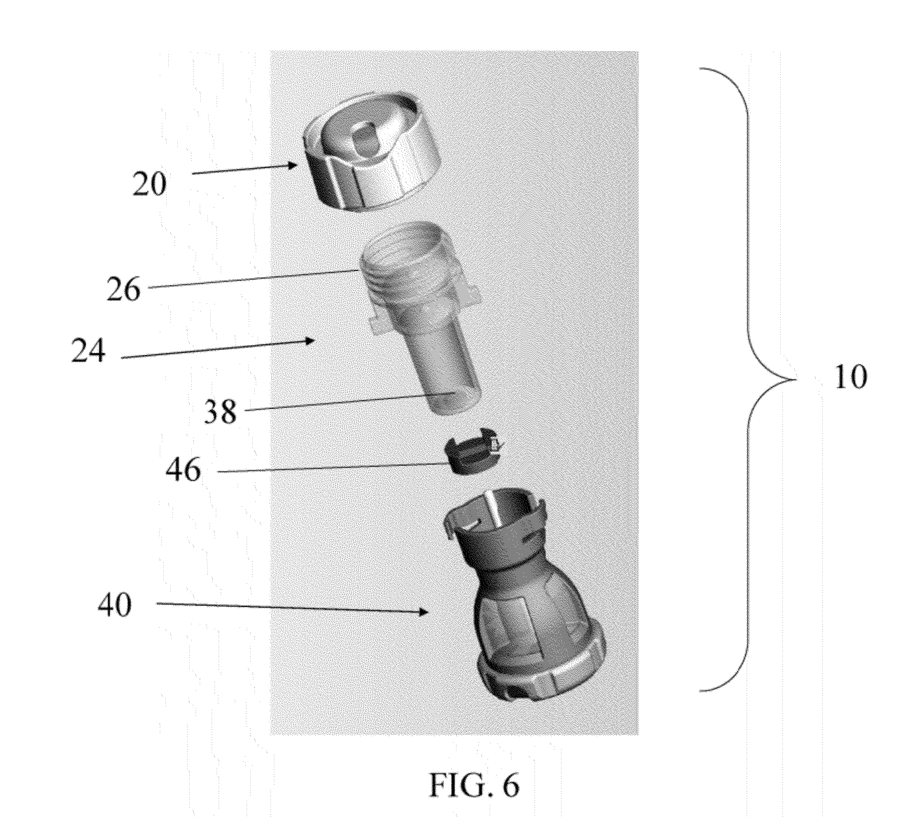

Device 10 comprises a lid 20, which covers a reagent tube 24, which sits inside of a base 40. Lid 20 and/or base 40 may be referred to by the generic term “cap.” A connector 46 secures reagent tube 24 inside base 40. Lid 20 may engage reagent tube 24 by way of screw threads 26 (as best seen in FIG. 6). Device 10 comprises two compartments heated by exothermic reactions, optionally moderated by PCMs. The first compartment, sample compartment 28, is heated to, for example, 95° C.-99° C. to prepare the sample for further processing. In one embodiment, device 10 can test a blood sample for malaria. In this embodiment, the sample preparation step lyses red blood cells and malaria parasites (releasing their DNA) and denaturing the DNA to facilitate the first step of LAMP (or other NA amplification technology if one is used in lieu of LAMP). However, device 10 can be used with other samples and other target pathogens. The second compartment, NA amplification (e.g., LAMP) compartment 36, is heated to, for example, 62° C.-65° C., (preferably at 62.5° C. for LAMP) for a set time limit, for example, approximately 60 minutes. This second heating facilitates the amplification stage (e.g., LAMP). A gasket 38 seals the bottom part of reagent tube 24.

Device 10 can be, for example, the size of a 0.5 ml cryo-vial and it contains all necessary reagents. In order to develop a fully-integrated device that is stable at ambient temperature, all NA amplification reagents (e.g., LAMP reagents) are in lyophilized (freeze-dried) form in the device, with the primers kept separate from the enzymes to avoid any reagent degradation prior to the start of the assay. As an example, lyophilization of active enzymes can be performed in the presence of a stabilizing sugar, such as trehalose or dextrose. As is shown in the Figures, the reagents may be kept in a separate compartment 32. However, in another embodiment the reagents may be kept in sample compartment 28, obviating the need for a separate compartment (not shown). The chemical heating reagents are contained within the lid 20 and the base 40.

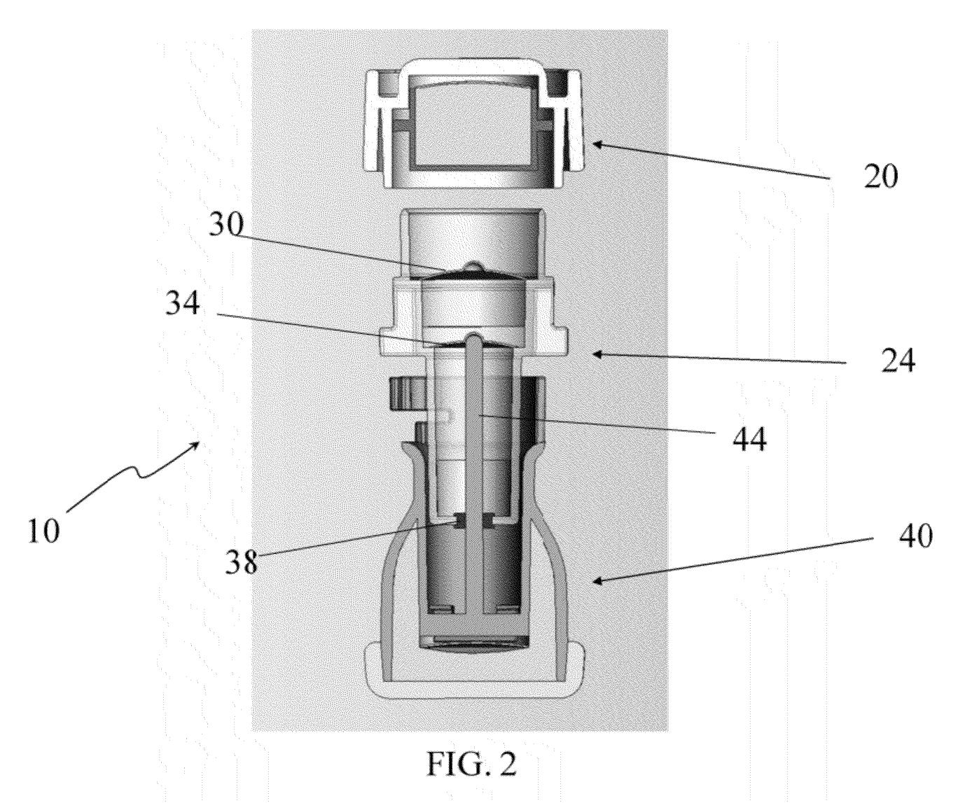

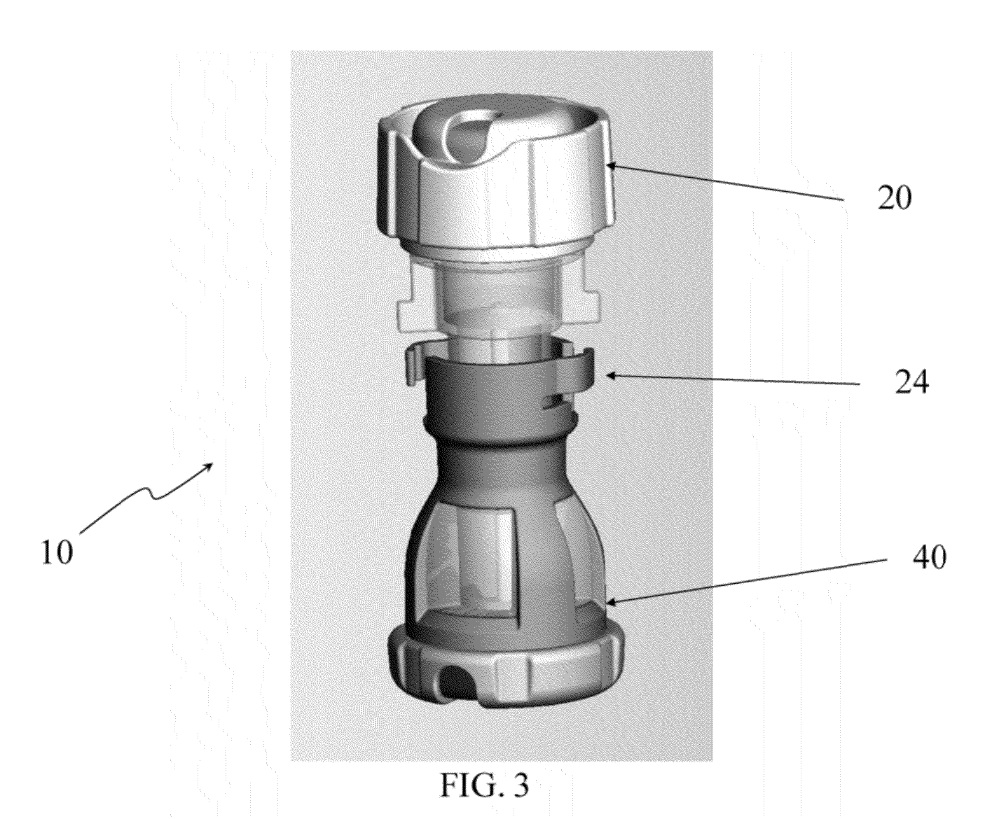

To begin a diagnostic test, a user will place a sample (e.g., a drop of blood) into the top of device 10 and screw on the lid 20. This will automatically initiate the first chemical heater (which may or may not include PCMs), which will be housed in lid 20. Other initiation means known in the art can be used in lieu of a screwing initiation, e.g., a clicker, shaking the device, bending a rod, adding water or other liquids, peeling off an adhesive to expose the chemicals to air, or any other means known in the art. Device 10 is shown in this initial configuration in FIGS. 2 and 3.

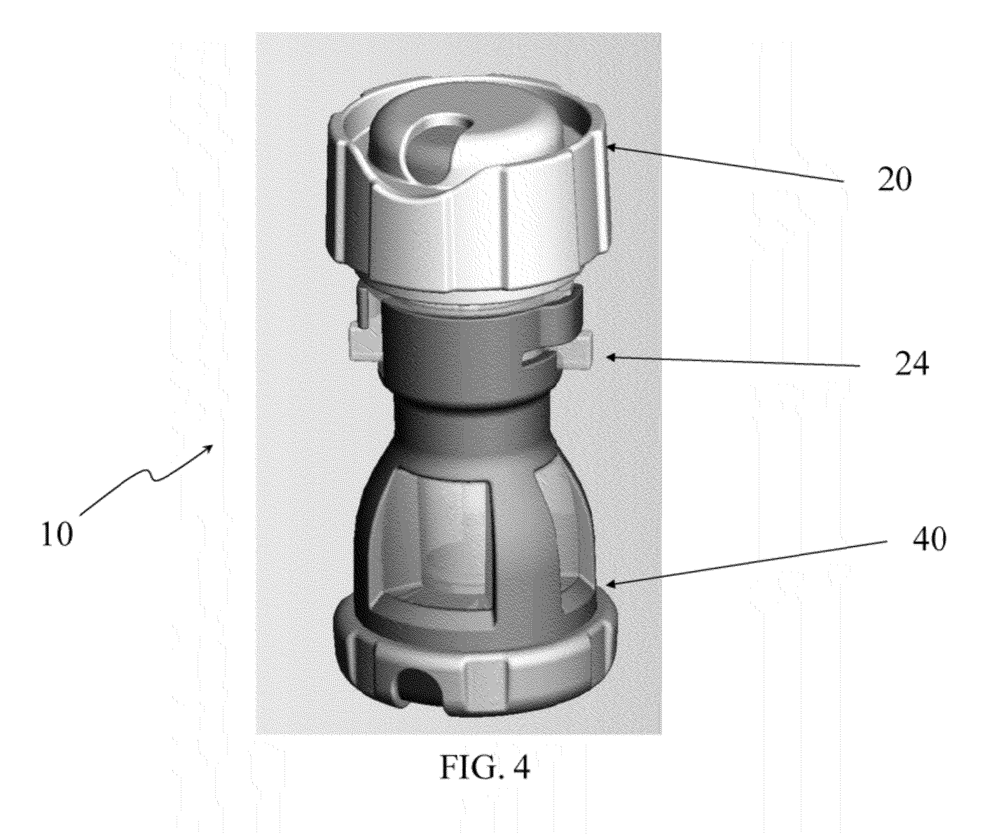

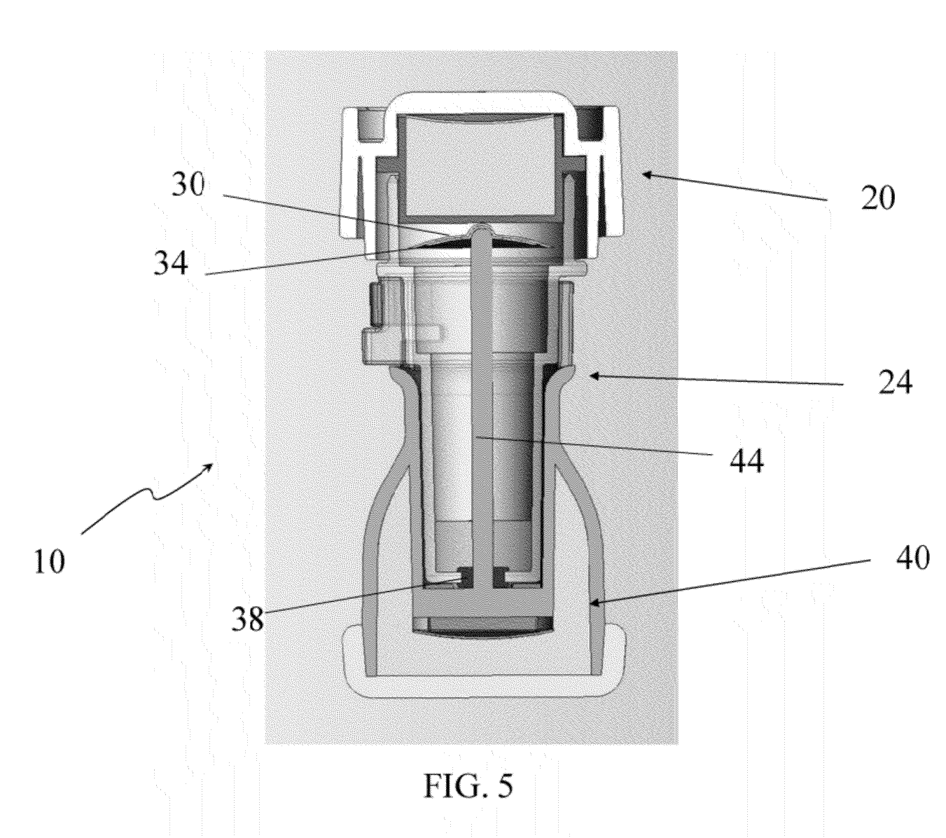

After a time (e.g., several minutes) and/or after a color temperature indicator 22 in lid 20 has indicated completion of the first step, the user will then compress the unit downwards, causing a spike 44 to puncture a membrane or membranes (sample membrane 30 and reagent membrane 34) thus allowing the reaction mixture to move to the NA amplification chamber 36 of device 10. Device 10 is shown in the compressed configuration in FIGS. 4 and 5. Other mechanisms could also be used to transfer the reaction mixture from sample compartment 28 to NA amplification chamber 36, as would be apparent to one of skill in the art. Device 10 is shown in the compressed configuration in FIGS. 4 and 5.

After the reaction mixture is transferred to NA amplification chamber 36, a second chemical heater (which also may or may not include PCMB) is activated, such as by turning bottom screw head 42. The second heating may also optionally have its own associated temperature indicator 48.

After some shaking for reagent mixing, and for example, approximately 30-60 minutes of heating in the second compartment, the user may then look through the window 50 in the base 40 of the device to observe for indicia of a positive reaction (e.g., turbidity of the reaction mixture or fluorescence, or both).

In another embodiment (not shown), device 10 can comprise a single exothermic chemical heater configured to provide a first temperature (e.g., 62-65° C.) for a first time interval (e.g., approximately 60 minutes) corresponding to sample preparation requirements and to provide a second temperature (e.g., 95-99° C.) for a second time interval (e.g., approximately 30-60 minutes) corresponding to a nucleic-acid amplification protocol. Chemical heaters capable of providing multiple temperature plateaus are discussed in U.S. patent application Ser. No. 12/134,965, filed Jun. 6, 2008, titled “CHEMICAL TEMPERATURE CONTROL,” the disclosure of which is incorporated herein by reference in its entirety. As one of ordinary skill in the art would recognize, these temperatures and time intervals are given by way of example; device 10 can be adapted to test for multiple sample types using multiple NA amplification protocols. Various sample types and/or NA amplification protocols may require different temperatures and/or time intervals.

As used herein, the phrases “lid” and “base” are not meant to limit the invention by implying a vertical orientation, but are merely used as a convenience. For example, if device 10 were inverted from the configuration depicted in the figures, 20 would still be the “lid,” while 40 would still be the “base.” As another example, device 10 could be tilted onto its side (from the position depicted in the figures), and 20 would still be the “lid,” while 40 would still be the “base.” Similarly, the term “cap” does not limit the present invention by implying a specific orientation.

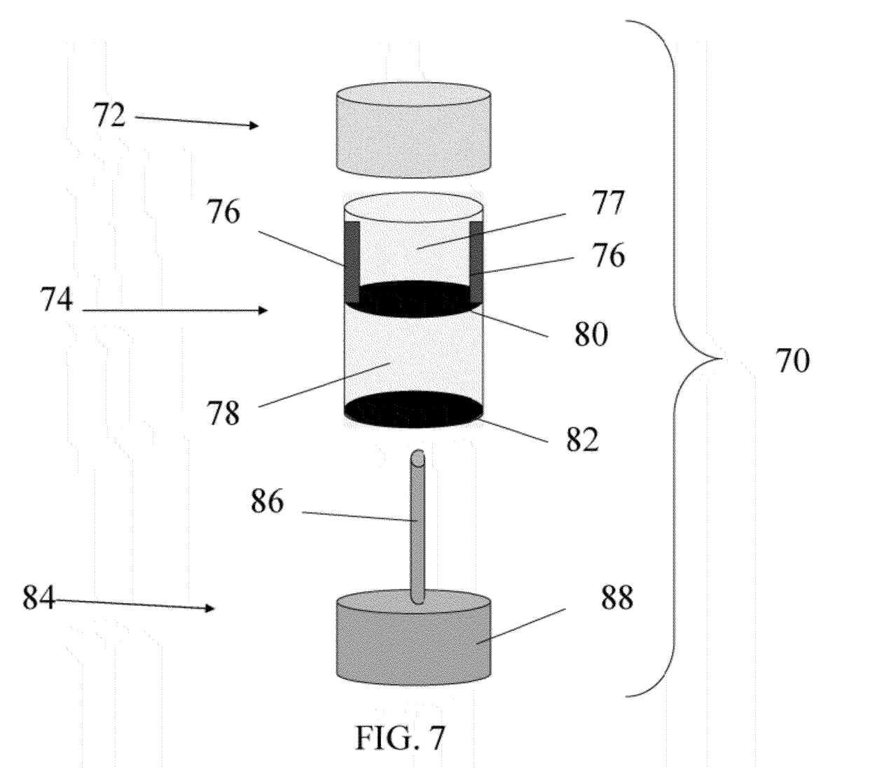

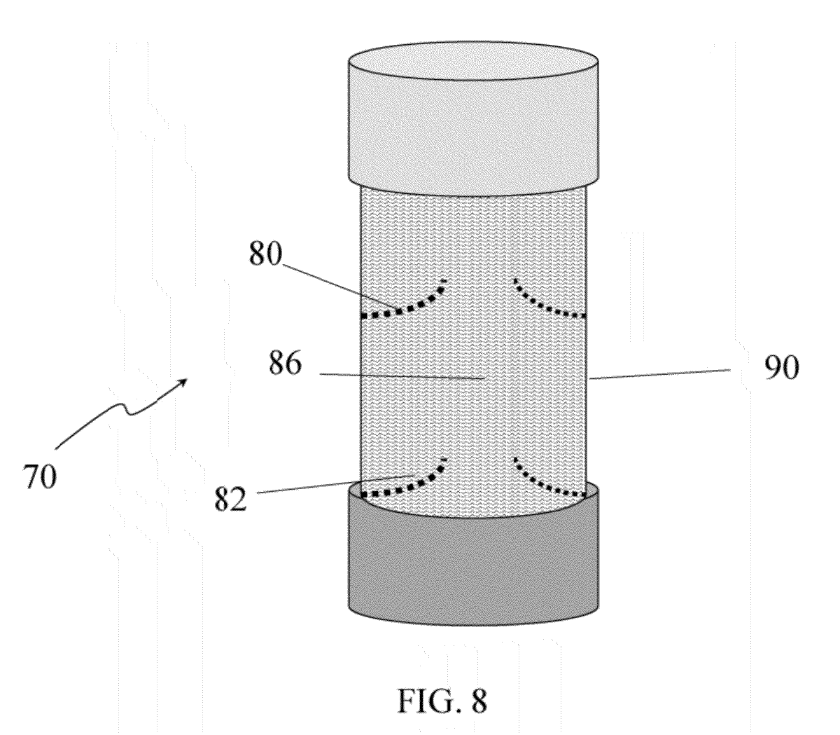

A schematic view of a self-contained, easy to use assay device, according to the present invention is shown generally at 70 in FIGS. 7 and 8. As shown, device 70 includes a top heat mixture 72 situated, a tube prepared with reagent and sample 74 and a bottom heat mixture 84. Top heat mixture 72 contains an exothermic chemical mix or PCMs in order to provide a temperature profile to prepare a sample for a NA amplification procedure. Bottom heat mixture 84 contains a second exothermic chemical mix or second PCMs in order to provide a second temperature profile suitable for a NA process (e.g., LAMP).

Reagents 76 are provided, for example in lyophilized (freeze-dried) form, in a top sample area 77 of tube 74, with the primers kept separate from the enzymes to avoid any reagent degradation prior to the start of the assay. A bottom part 78 of reagent tube 74 is provided for NA amplification. Bottom 84 includes a spike 86 which is used to rupture sample membrane 80 and NA membrane 82 at the initiation of the NA amplification process.

In operation, a user places a sample in sample compartment 77. A first exothermic reaction is initiated in 72 by any available means (a screw top, a clicker, shaking the device, bending a rod, adding water or other liquids, peeling off an adhesive to expose the chemicals to air, or any other means known in the art). At the conclusion of the sample heating phase, the user causes spike 86 to rupture membranes 80 and 82, letting the sample, reagents, and a buffer from compartment 78 mix together at the bottom of reagent tube 74. The user then causes heater 88 to initiate a second exothermic reaction to begin the process of NA amplification (e.g., LAMP). As seen in FIG. 8, device 70 includes a viewing area 90 so that the user can observe indicia (e.g., turbidity and/or fluorescence) of a positive test. FIG. 8 shows device 70 at the conclusion of a positive test. Note that membranes 80 and 82 are ruptured, and that the solution seen through viewing area 90 displays turbidity. Other indicia of positive test results can be employed, as is known in the art, for example fluorescence. In one embodiment, a fluorescent dye, e.g., an intercalating fluorescent dye, can be included. The NA amplicon can be detected as a value of fluorescence in real-time when there is an increase in fluorescence intensity caused by the dye. In some embodiments turbidity and fluorescence can simultaneously indicate a positive test result. To facilitate a fluorescent-based test, base 40 may include a fluorescent LED, a battery to power the LED, and a power switch to control the LED (not shown)

The heat mixtures 72 and 84 shown in FIG. 7 are depicted as being on ends of the assay device 70. Heat mixture 72 is referred to as a “top” heat mixer, while heat mixture 84 is referred to as a “bottom” heat mixture. As used herein, the phrases “top” and “bottom” are not meant to limit the invention. In other words, these phrases are not meant to imply a vertical orientation, but are merely used as a convenience. For example, if device 70 were inverted from the configuration depicted in the figures, heat mixture 72 would still be referred to as the “top” heat mixture. In an alternate embodiment, heaters could be situated annually around the tube instead of being disposed on ends of the tube (as shown in FIGS. 7-8), as explained below.

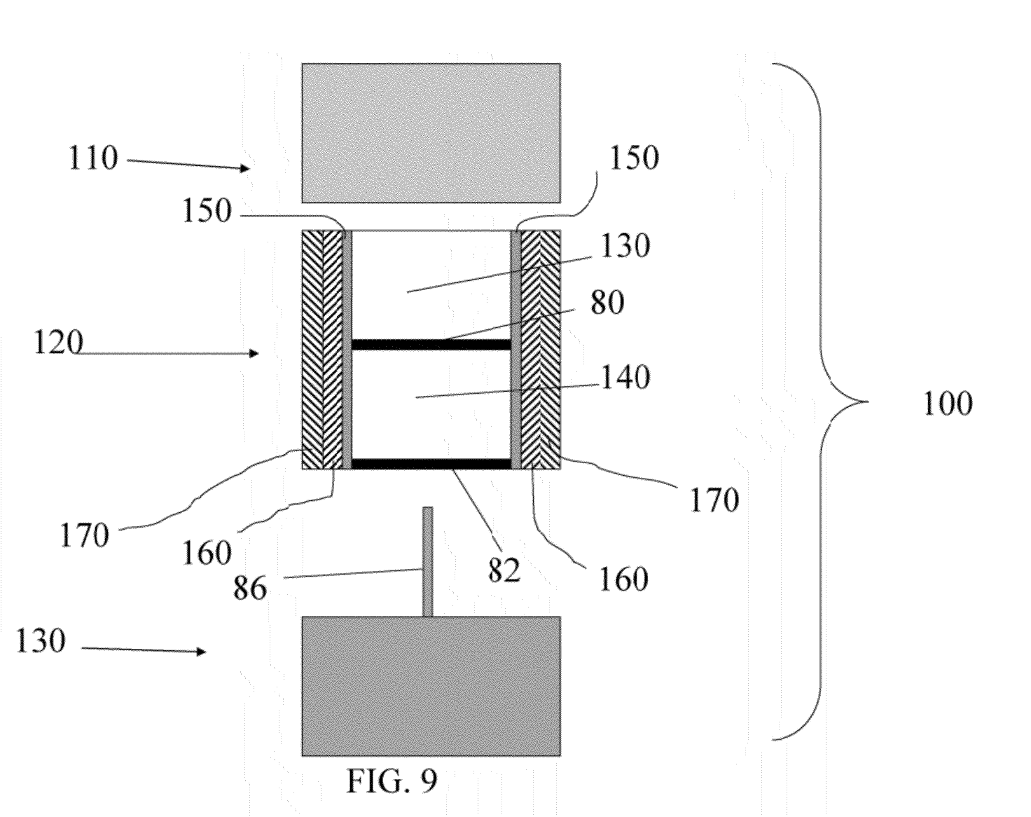

FIG. 9 shows an exploded cross-sectional view of a self-contained, easy to use assay device 100, according to the present invention. Device 100 features a lid 110 disposed on a tube 120. Tube 120 is disposed on a base 130. In this embodiment, the heaters are not in the lid 110 or base 130; instead, they are situated annually around tube 120. A first part of tube 120 is a sample compartment 130 which is disposed adjacent to a NA amplification compartment 140. Sample compartment 130 may have reagents contained therein, while amplification compartment 140 may have a buffer therein. PCM 150 is placed directly outside of the sample compartment 130 and amplification compartment 140. This placement puts the PCM in thermal contact with tube 120 in order to control its temperature. PCM 150 can use a first PCM for sample compartment 130 and a second PCM for amplification compartment 140 in order to provide for two different temperatures (for example a sample preparation temperature and a NA amplification temperature). Exothermic chemical mixture 160 is placed just outside of PCM 150 to provide a heat input which causes the PCM to assume its phase change temperature, which in turn imposes a well-defined temperature to tube 120.

The mixture can employ a first exothermic chemical for sample compartment 130 and a second exothermic chemical for amplification compartment 140 if desired (not shown), or a single exothermic chemical mixture can be provide as shown in the illustrated embodiment. Exothermic chemical mixture can be initiated when lid 110 is screwed onto or placed onto tube 120. Other initiation means known in the art can be used in lieu of a screwing initiation, e.g., a clicker, shaking the device, bending a rod, adding water or other liquids, peeling off an adhesive to expose the chemicals to air, or any other means known in the art. Insulation 170 is placed immediately outside of exothermic chemical mixture 160 in order to minimize heat losses to the outside.

Although shown only in FIG. 9 (at 170), insulation may be used in any of the various embodiments of the present invention. Urethane foam, besides being an excellent insulator, is also cheap and easy to incorporate into various devices. Alternately, any material having a relatively low heat transfer coefficient may be used. Since heat transfer is a surface phenomena, it is also advantageous to use geometries having low surface to volume ratios, for example, spherical or cylindrical geometries. Insulators and geometry should be used to best advantage whenever temperature and/or heat flux is to be controlled (i.e., in NA amplification applications such as LAMP).

Membranes 30 and 34 discussed above with reference to FIGS. 1-6 and membranes 80 and 82 discussed above with reference to FIGS. 7-9 may be ruptured by spikes 44 and 86, respectively. In embodiments, frangible membranes can be used which are capable of being ruptured by a sharp object or even air or liquid pressure (not shown). Frangible blisters capable of being selectively ruptured in order to release fluids contained within them are disclosed in U.S. Provisional Patent Application No. 60/981,783, filed Oct. 22, 2007, titled “MANUALLY CONTROLLABLE FLUIDIC NETWORK,” the disclosure of which is hereby incorporated in its entirety by reference thereto.

Since embodiments of the present invention utilize chemical heaters, there is a possibility that gases, vapors, or steam may be produced. To compensate for this possibility, in one embodiment, vents (not shown) may be added to the devices of the present invention. In another embodiment, the devices of the present invention can be expandable such that the internal pressure of the devices does not exceed some threshold level when gases, vapors, or steam is produced. In another embodiment, check valves (not shown) may be added to the devices of the present invention. In another embodiment, the devices of the present invention may be expandable and also may have vents and/or check valves. There are various other pressure limiting devices known in the art which can be used with the present invention to limit or control internal pressures due to gases, vapors, or steam which may be produced during either the sample preparation or NA amplification steps.

Using the present invention, highly sensitive assays for pathogens can be performed by minimally trained personnel in developing countries or, in case of outbreaks (intentional or natural) by first responders. Home tests for genetic markers or infectious diseases may become possible, and forensic assays might be greatly accelerated and simplified. As with many other technologies that have spread into common use, the potential impact is enormous.

As an example, an assay based on the present invention could be configured as a home test that would allow untrained home users to differentiate between the common cold and influenza. If influenza is identified, they could then seek medical care and antiviral drugs. The proposed assay has the potential to identify the presence of a pathogen on nasal swabs within 30-60 minutes, with much greater sensitivity than currently available, relatively insensitive rapid assays.

Even more important, in the case of a looming threat of a pandemic strain of influenza or other virus, or an intentional release of a pathogen, an assay according to the present invention could be distributed to drug stores or public health facilities for use by home users or local health care workers. Again, this would allow patients or their caregivers to distinguish a serious infection from the background of relatively harmless common infections. Local or home diagnosis could have a huge impact by alleviating demand on higher-level medical facilities that may already be stretched beyond their limits in such situations. Those same assays could also be made available to, and used by, first responders or border personnel during a pandemic or biothreat crisis.

Furthermore, for diseases that carry a stigma, such as many sexually transmitted diseases, many patients in both developed and developing countries may prefer assays that provide answers in the privacy of their homes. Currently available assays that fit this niche are rapid strip assays, which detect the immune response to a pathogen, or pathogen-specific antigens; however, they are several orders of magnitude less sensitive than NA-based assays. The assay according to the present invention is significantly more sensitive and specific than current rapid strip tests, and therefore is able to detect an infection much earlier, ideally at the very onset of symptoms.

Another application for the assay according to the present invention is the detection of genetic markers for specific diseases. Such markers hold great promise because they can predict an individual's susceptibility to developing inherited metabolic diseases and some cancers. This allows patients to lower those risks through lifestyle adjustments and medical consultation. Again, patients worried about privacy issues may opt for a test that they can carry out themselves, in their homes, rather than relying on medical providers to guard their medical records.

Complexity, rapidity, equipment requirements, and cost determine how close a technology can be brought to individuals. The conversion of a complex technology such as the NA amplification assay to something akin to a pregnancy test will change the way these tests are utilized.

The foregoing description of the embodiments are presented for purposes of illustration and description. The description is not intended to be exhaustive or to limit the invention to the precise form disclosed. Modifications and variations are possible in light of the above teachings, as would be apparent to one of skill in the art. For example, devices 10 or 70 can be different shapes, have a different number of compartments, use different exothermic chemicals and/or PCMs and can operate on different samples testing for various target pathogens.

While this invention has been particularly shown and described with reference to preferred embodiments thereof, it will be understood by those skilled in the relevant art(s) that various changes in form and details may be made therein without departing form the spirit and scope of the invention. Thus, the breadth and scope of the present invention should not be limited by any of the above-described exemplary embodiments, but should be defined only in accordance with the following claims and their equivalents.

What is claimed is:

1. An assay platform, comprising:
a main body having a first end and a second end;
a first cap detachably connected to said first end of said main body, wherein said first cap comprises a first exothermic chemical heater; and
a second cap detachably connected to said second end of said main body, wherein said second cap comprises a second exothermic chemical heater;
wherein said main body further comprises a first compartment for sample preparation and a second compartment for nucleic-acid amplification.

2. The assay platform according to claim 1, wherein said main body is tube-like.

3. The assay platform according to claim 1, wherein said main body comprises a vial.

4. The assay platform according to claim 3, wherein said main body is a sample tube.

5. The assay platform according to claim 1, wherein said first exothermic chemical heater is initiated when said first cap is connected to said main body.

6. The assay platform according to claim 1, wherein said second exothermic chemical heater is initiated when said second cap is moved relative to said main body.

7. The assay platform according to claim 6, wherein said second exothermic chemical heater is initiated when said second cap and said main body are compressed.

8. The assay platform according to claim 1, wherein said first compartment and said second compartment are separated by a membrane.

9. The assay platform according to claim 8, wherein said base further comprises a spike, wherein said spike is capable of puncturing said membrane when said second cap is moved relative to said main body.

10. The assay platform according to claim 9, wherein said spike is capable of puncturing said membrane when said second cap and said main body are compressed.

11. The assay platform according to claim 1, wherein said nucleic-acid amplification is loop-mediated isothermal amplification.

12. The assay platform according to claim 1, wherein said second cap further comprises a window allowing observation of said nucleic-acid amplification.

13. The assay platform according to claim 1, wherein said first compartment comprises a reagent mixture for said sample preparation.

14. The assay platform according to claim 1, further comprising:
an elongated member capable of being received in said second end of said main body;
wherein said main body further comprises a membrane separating said sample preparation compartment from said nucleic-acid amplification compartment;
wherein said first exothermic chemical heater is initiated when said first cap is connected to said sample tube and said second exothermic chemical heater is initiated when said second cap and said sample tube are compressed; and
wherein said elongated member is capable of puncturing said membrane when said second cap and said sample tube are compressed.

15. An assay platform, comprising:
a main body having a first end and a second end;
a cap detachably connected to said first end of said main body, wherein said cap comprises an exothermic chemical heater and phase change material;
wherein said main body further comprises a first compartment for sample preparation and a second compartment for nucleic-acid amplification;
wherein said exothermic chemical heater and said phase change material provide a first temperature for a first time interval corresponding to a sample preparation temperature and time interval; and
wherein said exothermic chemical heater provides a second temperature for a second time interval corresponding to a nucleic-acid amplification temperature and time interval.

16. The assay platform according to claim 15, wherein said exothermic chemical
heater is initiated when said cap is connected to said main body.

17. The assay platform according to claim 16, wherein said main body comprises a
tube or vial.

18. An assay platform, comprising:
a main body having a first compartment for sample preparation and a second compartment for nucleic-acid amplification;
a first phase change material characterized by a first phase change temperature surrounding at least a portion of said first compartment;
a second phase change material characterized by a second phase change temperature surrounding at least a portion of said second compartment; and
an exothermic chemical heater in thermal contact with said first and second phase change materials.

19. The assay platform according to claim 18, further comprising a membrane separating said first and second compartments.

20. The assay platform according to claim 18, further comprising insulation surrounding at least a portion of said exothermic chemical heater.
